(19) United States
(12) Patent Application Publication
(10) Pub. No.: US 2026/0279544 A1
(43) Pub. Date: Sep. 17, 2026

(54) INFORMATION PROCESSING METHOD, RECORDING MEDIUM, INFORMATION PROCESSING SYSTEM, AND LEARNING SYSTEM

(71) Applicant: Panasonic Intellectual Property Management Co., Ltd., Osaka (JP)

(72) Inventors: KAORI SUZUKI, Osaka (JP); KENJI KONDO, Kyoto (JP); KIYOTAKA TSUJI, Osaka (JP)

(21) Appl. No.: 19/680,368

(22) Filed: May 18, 2026

Related U.S. Application Data
(63) Continuation of application No. PCT/JP2024/042717, filed on Dec. 3, 2024.

(30) Foreign Application Priority Data
Dec. 11, 2023 (JP) 2023-208653

(51) Int. Cl. G16H 20/70; G16H 20/60; G16H 50/30

(52) U.S. Cl. CPC G16H 20/70; G16H 20/60; G16H 50/30

(57) ABSTRACT

An information processing method is an information processing method performed by a computer, and includes a step of acquiring desired emotion information concerning an emotion which a user desires to achieve through a meal, a step of generating recommended meal information concerning content of a meal recommended for the user to achieve the emotion which the user desires to achieve through the meal on the basis of the desired emotion information, and a step of outputting the recommended meal information thus generated.

### BACKGROUND

### 1. Technical Field

The present disclosure relates to an information processing method, a recording medium, an information processing system, and a learning system.

### 2. Description of the Related Art

Japanese Unexamined Patent Application Publication No. 2022-150207 (hereinafter referred to as Patent Literature 1) discloses a recipe suggestion system that predicts a user's emotion in the future on the basis of user's schedule information and suggests a cooking recipe optimum for the predicted emotion.

### SUMMARY

However, according to the above conventional technique, it is difficult to predict a user's emotion in the future on the basis of user's schedule information, and it is therefore difficult to provide recommended meal information for achieving an emotion desired by the user.

One non-limiting and exemplary embodiment provides an information processing method and others that can provide recommended meal information for achieving an emotion desired by a user.

In one general aspect, the techniques disclosed here feature an information processing method performed by a computer, the information processing method including acquiring desired emotion information concerning an emotion which a user desires to achieve through a meal, generating recommended meal information concerning content of a meal recommended for the user to achieve the emotion which the user desires to achieve through the meal on the basis of the desired emotion information, and outputting the recommended meal information thus generated.

According to the present disclosure, it is possible to provide recommended meal information for achieving an emotion desired by a user.

It should be noted that general or specific embodiments may be implemented as an apparatus, a system, an integrated circuit, a computer program, a computer-readable recording medium, or any selective combination thereof. Examples of the computer-readable recording medium include a non-volatile recording medium such as a Compact Disc-Read Only memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating a configuration of a learning system and a configuration of an information processing system according to Embodiment 1;

FIG. 2 is a sequence diagram of a learning phase according to Embodiment 1;

FIG. 3 illustrates an example of first emotion information according to Embodiment 1;

FIG. 4 illustrates an example of first biological information according to Embodiment 1;

FIG. 5 illustrates an example of meal information according to Embodiment 1;

FIG. 6 illustrates an example of second emotion information according to Embodiment 1;

FIG. 7 is a sequence diagram of a prediction phase according to Embodiment 1;

FIG. 8 illustrates an example of an input screen for desired emotion information according to Embodiment 1;

FIG. 9 illustrates an example of recommended meal information according to Embodiment 1;

FIG. 10 illustrates an example of a display screen for displaying recommended meal information according to Embodiment 1;

FIG. 11 is a block diagram illustrating a configuration of an information processing system and a configuration of a learning system according to Embodiment 2;

FIG. 12 is a sequence diagram of a learning phase according to Embodiment 2;

FIG. 13 illustrates an example of second biological information according to Embodiment 2;

FIG. 14 is a sequence diagram of a prediction phase according to Embodiment 2; and

FIG. 15 illustrates an example of a training dataset according to a modification.

### DETAILED DESCRIPTIONS

### Outline of Present Disclosure

An outline of the present disclosure is described before description of embodiments.

An information processing method according to a first aspect of the present disclosure is an information processing method performed by a computer, and includes acquiring desired emotion information concerning an emotion which a user desires to achieve through a meal, generating recommended meal information concerning content of a meal recommended for the user to achieve the emotion which the user desires to achieve through the meal on the basis of the desired emotion information, and outputting the recommended meal information thus generated.

According to this, recommended meal information for achieving an emotion desired by a user is generated on the basis of desired emotion information concerning the emotion which the user desires to achieve through a meal. Therefore, more suitable recommended meal information can be provided to achieve the emotion desired by the user. For example, in a case where meal information is provided on the basis of a user's emotion in the future predicted from schedule information as in Patent Literature 1, there is a possibility that meal information is provided on the basis of an emotion different from an emotion desired by the user. Therefore, according to the method described in Patent Literature 1, meal information unsuitable for achieving an emotion desired by a user may be undesirably provided. On the other hand, according to the information processing method according to the first aspect of the present disclosure, recommended meal information is generated on the basis of desired emotion information, and it is therefore possible to provide more suitable recommended meal information to achieve an emotion desired by a user. Furthermore, it is possible to enhance a customer experience value for a user to whom the recommended meal information is provided since it is possible to achieve an emotion which the user desires to achieve through a meal.

An information processing method according to a second aspect of the present disclosure is the information processing method according to the first aspect in which in the generating the recommended meal information, the recommended meal information is generated by inputting the desired emotion information to a trained model, and the trained model has been trained by machine learning using a set of meal information concerning content of a meal eaten by the user and second emotion information concerning an emotion of the user after the meal as a training dataset.

According to this, recommended meal information is generated by inputting desired emotion information to a trained model that has been trained by machine learning using a set of information related to a user as a training dataset, and it is therefore possible to provide more suitable recommended meal information to the user.

An information processing method according to a third aspect of the present disclosure is the information processing method according to the first aspect, further including acquiring at least one of first emotion information concerning an emotion of the user before the meal or first biological information of the user before the meal, in which in the generating the recommended meal information, the recommended meal information is generated on the basis of at least one of the first emotion information or the first biological information in addition to the desired emotion information.

According to this, recommended meal information is generated on the basis of an emotion and/or biological information of a user before a meal in addition to an emotion which the user desires to achieve through a meal, and it is therefore possible to provide more suitable recommended meal information to achieve the emotion desired by the user. In particular, it is possible to provide more suitable recommended meal information to change a user's state before the meal to a user's state after the meal for achieving an emotion desired by the user.

An information processing method according to a fourth aspect of the present disclosure is the information processing method according to the third aspect, in which in the generating the recommended meal information, the recommended meal information is generated by inputting the desired emotion information and at least one of the first emotion information or the first biological information to a trained model, and the trained model has been trained by machine learning using a set of meal information concerning content of a meal eaten by the user, second emotion information concerning an emotion of the user after the meal, and at least one of first emotion information concerning an emotion of the user before the meal or first biological information of the user before the meal as a training dataset.

According to this, recommended meal information is generated by inputting desired emotion information to a trained model that has been trained by machine learning using a set of information related to a user as a training dataset, and it is therefore possible to provide more suitable recommended meal information to the user.

An information processing method according to a fifth aspect of the present disclosure is the information processing method according to the first aspect, in which the generating the recommended meal information includes generating desired biological information corresponding to the emotion which the user desires to achieve through the meal on the basis of the desired emotion information and generating the recommended meal information on the basis of the desired biological information.

According to this, desired biological information is generated on the basis of desired emotion information, and recommended meal information is generated on the basis of the desired biological information. By thus generating desired biological information from desired emotion information and then generating recommended meal information from the desired biological information, not only an emotion consciously desired by a user, but also an emotion subconsciously desired by the user can be reflected in the recommended meal information, and recommended meal information more suitable for an emotion desired by the user can be provided.

An information processing method according to a sixth aspect of the present disclosure is the information processing method according to the fifth aspect, in which in the acquiring the desired biological information, the desired biological information is generated by inputting the desired emotion information to a first trained model, and the first trained model has been trained by machine learning using a set of emotion information concerning an emotion of the user and biological information of the user corresponding to the emotion information as a training dataset.

According to this, desired biological information is generated by inputting desired emotion information to a first trained model that has been trained by machine learning using a set of information related to a user as a training dataset, and it is therefore possible to generate biological information of the user corresponding to an emotion desired by the user.

An information processing method according to a seventh aspect of the present disclosure is the information processing method according to the fifth or sixth aspect, in which in the acquiring the recommended meal information, the recommended meal information is generated by inputting the desired biological information to a second trained model, and the second trained model has been trained by machine learning using a set of meal information concerning content of a meal eaten by the user and second biological information of the user after the meal as a training dataset.

According to this, recommended meal information is generated by inputting desired biological information to a second trained model that has been trained by machine learning using a set of information related to a user as a training dataset, and it is therefore possible to provide more suitable recommended meal information to the user.

An information processing method according to an eighth aspect of the present disclosure is the information processing method according to the fifth or sixth aspect, further including acquiring at least one of first emotion information concerning an emotion of the user before the meal or first biological information of the user before the meal, in which in the generating the recommended meal information, the recommended meal information is generated on the basis of at least one of the first emotion information or the first biological information in addition to the desired biological information.

According to this, recommended meal information is generated on the basis of an emotion and/or biological information of a user before a meal in addition to an emotion which a user desires to achieve through a meal, and it is therefore possible to provide more suitable recommended meal information to achieve the emotion desired by the user. In particular, it is possible to provide more suitable recommended meal information to change a user's state before a meal to a user's state after the meal for achieving an emotion desired by the user.

An information processing method according to a ninth aspect of the present disclosure is the information processing method according to the eighth aspect, in which in the generating the recommended meal information, the recommended meal information is generated by inputting the desired biological information and at least one of the first emotion information or the first biological information to a second trained model, and the second trained model has been trained by machine learning using a set of meal information concerning content of a meal eaten by the user, second biological information of the user after the meal, and at least one of first emotion information concerning an emotion of the user before the meal or first biological information of the user before the meal as a training dataset.

According to this, recommended meal information is generated by inputting desired biological information to a second trained model that has been trained by machine learning using a set of information related to a user as a training dataset, and it is therefore possible to provide more suitable recommended meal information to the user.

An information processing method according to a tenth aspect of the present disclosure is the information processing method according to any one of the first to ninth aspects, in which in the outputting the recommended meal information, a recommended amount of each of dish categories that constitute the meal is displayed as the recommended meal information on a display.

According to this, a recommended amount for each dish category can be presented on a display, and user's meal preparation can be effectively supported.

An information processing method according to an eleventh aspect of the present disclosure is the information processing method according to the tenth aspect, in which in the outputting the recommended meal information, one or more dish menu candidates belonging to each of the dish categories are displayed, and the information processing method further includes receiving, from the user, selection of the displayed one or more dish menu candidates and input of an amount of each of the selected one or more dish menus.

According to this, a dish menu that satisfies a recommended amount for each dish category indicated by recommended meal information can be selected from among displayed candidates, and user's meal preparation can be more effectively supported.

An information processing program according to a twelfth aspect of the present disclosure causes a computer to perform the information processing method according to any one of the first to eleventh aspects.

According to this, effects similar to those of the information processing method can be realized by the information processing program.

An information processing system according to a thirteenth aspect of the present disclosure includes an acquirer that acquires desired emotion information concerning an emotion which a user desires to achieve through a meal; a generator that generates recommended meal information concerning content of a meal recommended for the user to achieve the emotion which the user desires to achieve through the meal on the basis of the desired emotion information; and an outputter that outputs the recommended meal information thus generated.

According to this, effects similar to those of the information processing method can be realized by the information processing system.

A learning system according to a fourteenth aspect of the present disclosure includes a meal information acquirer that acquires meal information concerning content of a meal eaten by a user; a biological information acquirer that acquires biological information of the user after the meal; an emotion information acquirer that acquires emotion information concerning an emotion of the user after the meal; a first model constructor that constructs a first trained model that outputs desired biological information corresponding to desired emotion information concerning an emotion which the user desires to achieve through a meal on the basis of input of the desired emotion information by performing machine learning using a set of the emotion information and the biological information as a training dataset; and a second model constructor that constructs a second trained model that outputs recommended meal information recommended for the user to achieve the desired biological information through a meal on the basis of input of the desired biological information by performing machine learning using a set of the biological information and the meal information as a training dataset.

According to this, a first trained model that generates desired biological information upon input of desired emotion information and a second trained model that generates recommended meal information upon input of desired biological information are constructed. Desired biological information is generated from desired emotion information by using the first trained model, and recommended meal information is generated from the desired biological information by using the second trained model. By thus generating desired biological information from desired emotion information and generating recommended meal information from the desired biological information, an emotion subconsciously desired by a user can also be reflected in the desired biological information, and it is therefore possible to provide recommended meal information more suitable for an emotion desired by the user.

Embodiments are specifically described below with reference to the drawings.

Each of the embodiments described below illustrates a general or specific example. Numerical values, shapes, materials, constituent elements, the way in which the constituent elements are disposed and connected, steps, the order of steps, and the like in the embodiments below are examples and do not limit the technique of the present disclosure. Each drawing is a schematic view and is not necessarily strict illustration. Therefore, for example, scales and the like in the drawings do not necessarily match. In the drawings, substantially identical constituent elements are given identical reference signs, and repeated description thereof may be omitted or simplified.

In the following description, all or a part of any of circuit, unit, device, part or portion, or any of functional blocks in the block diagrams may be implemented by executing a software program. In such a case, the software program is recorded on one or more non-transitory recording media such as a ROM, an optical disk or a hard disk drive, and when the software program is executed by a processor, the software program causes the processor together with peripheral devices to execute the functions specified in the software program. A system or apparatus may include such one or more non-transitory recording media on which the software program is recorded and a processor together with necessary hardware devices such as a memory and an interface.

Furthermore, all or a part of any of circuit, unit, device, part or portion, or any of functional blocks in the block diagrams may be, for example, implemented as one or more of electronic circuits including a semiconductor device, a semiconductor integrated circuit (IC) or a large scale integration (LSI). The LSI or IC can be integrated into one chip, or also can be a combination of chips. For example, functional blocks other than a memory may be integrated into one chip. The name used here is LSI or IC, but it may also be called system LSI, very large scale integration (VLSI), or ultra large scale integration (ULSI) depending on the degree of integration. A Field Programmable Gate Array (FPGA) that can be programmed after manufacturing an LSI or a reconfigurable logic device (RLD) that allows reconfiguration of the connection or setup of circuit cells inside the LSI can be used for the same purpose.

### Embodiment 1

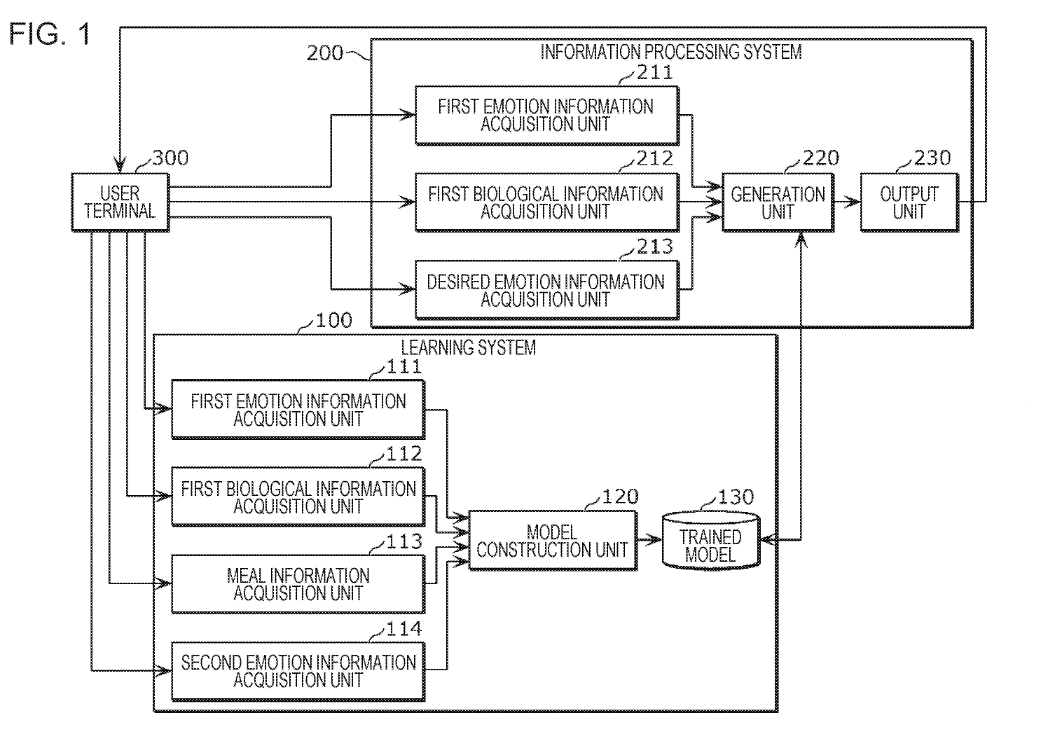

A learning system 100 and an information processing system 200 according to Embodiment 1 are described below with reference to FIG. 1. FIG. 1 is a block diagram illustrating a configuration of the learning system 100 and a configuration of the information processing system 200 according to the present embodiment.

### 1.1 Configuration of Learning System 100

The learning system 100 is realized by using one or more computers (e.g., an application server and a database server). The one or more computers may be provided by cloud computing.

As illustrated in FIG. 1, the learning system 100 includes a first emotion information acquisition unit 111, a first biological information acquisition unit 112, a meal information acquisition unit 113, a second emotion information acquisition unit 114, a model construction unit 120, and a trained model 130.

The first emotion information acquisition unit 111 can acquire first emotion information concerning a user's emotion before a meal from a user terminal 300. The first emotion information indicates a user's mental attribute before a meal. For example, the first emotion information indicates a mental attribute selected by the user from among predetermined mental attributes. Examples of the predetermined mental attributes include excited, energetic, happy, joyful, enjoyable, satisfied, relieved, relaxed, tired, bored, depressed, sad, displeased, worried, afraid, and vigilant. The first emotion information may be, for example, expressed by a coordinate value on a two-dimensional plane constituted by two axes, specifically, an axis indicating comfortable/uncomfortable and an axis indicating wakeful/sleepy. Note that the first emotion information may be a mental attribute based on a result of emotion assessment conducted on the user or may be a mental attribute detected and recognized by using emotion detection and recognition technique. Note that the first emotion information acquisition unit 111 is optional and need not be included in the learning system 100.

The first biological information acquisition unit 112 can acquire first biological information of the user before a meal from the user terminal 300. Note that the first biological information acquisition unit 112 may acquire the first biological information from a biological sensor attached to the user. The first biological information is physiological information which the user exhibits before the meal. The first biological information can be, for example, a heart rate, a body temperature, a blood pressure, blood sugar, or any combination thereof, but is not limited to these. Note that the first biological information acquisition unit 112 is optional and need not be included in the learning system 100.

The meal information acquisition unit 113 can acquire meal information concerning content of a meal eaten by the user from the user terminal 300. The meal information indicates amounts of dish categories that constitute the meal. The dish categories can be, for example, categories (grain dishes, vegetable dishes, fish and meat dishes, milk products, and fruits) described in "Japanese Food Guide", [online], Ministry of Agriculture, Forestry and Fisheries, [retrieved on October 27, 2023], the Internet <URL: https://www.maff.go.jp/j/balance_guide/>, but are not limited to these. Note that nutritional components (e.g., carbohydrates, proteins, calcium, and vitamins) may be used instead of or in addition to the dish categories.

The second emotion information acquisition unit 114 can acquire second emotion information concerning a user's emotion after a meal from the user terminal 300. The second emotion information indicates a user's mental attribute after the meal. The second emotion information is similar to the first emotion information except for that the second emotion information indicates a user's mental attribute after a meal.

The model construction unit 120 can construct the trained model 130 by performing machine learning (supervised learning) using a set of the first emotion information, the first biological information, the meal information, and the second emotion information as a training dataset. The model construction unit 120 can improve prediction accuracy of the trained model 130 by repeating update of model parameters and evaluation of the model having the updated parameters by using training datasets. Note that the first emotion information and/or the first biological information are optional and need not be used for construction of the trained model 130. An algorithm used for construction of the trained model 130 is not limited in particular.

The trained model 130 is trained by machine learning using a set of the meal information concerning content of a meal eaten by the user, the second emotion information concerning a user's emotion after the meal, the first emotion information concerning a user's emotion before the meal, and the first biological information of the user before the meal as a training dataset. As a result, the trained model 130 can output recommended meal information concerning content of a meal recommended for the user to achieve an emotion which the user desires to achieve through a meal in response to input of desired emotion information concerning the emotion which the user desires to achieve through the meal, the first emotion information (optional) of the user before the meal, and the first biological information (optional) of the user before the meal. That is, in the trained model 130, the desired emotion information, the first emotion information (optional), and the first biological information (optional) are explanatory variables, and the recommended meal information is an objective variable.

### 1.2 Configuration of Information Processing System 200

The information processing system 200 is realized by using one or more computers (e.g., an application server and a database server). A part or all of the information processing system 200 may be configured by the same computer as the learning system 100.

As illustrated in FIG. 1, the information processing system 200 includes a first emotion information acquisition unit 211, a first biological information acquisition unit 212, a desired emotion information acquisition unit 213, a generation unit 220, and an output unit 230.

The first emotion information acquisition unit 211 can acquire the first emotion information from the user terminal 300, as with the first emotion information acquisition unit 111. Note that the first emotion information acquisition unit 211 is optional and need not be included in the information processing system 200.

The first biological information acquisition unit 212 can acquire the first biological information from the user terminal 300, as with the first biological information acquisition unit 112. Note that the first biological information acquisition unit 212 is optional and need not be included in the information processing system 200.

The desired emotion information acquisition unit 213 can acquire desired emotion information concerning an emotion which the user desires to achieve through a meal from the user terminal 300 before the meal. The desired emotion information indicates a user's mental attribute which the user desires to achieve through the meal. For example, the desired emotion information may be interpreted as information concerning an emotion which the user desires after the meal.

The generation unit 220 can generate the recommended meal information on the basis of the first emotion information, the first biological information, and the desired emotion information. Specifically, the generation unit 220 can generate the recommended meal information by inputting the first emotion information, the first biological information, and the desired emotion information to the trained model 130. Note that in the generation unit 220, generation of the recommended meal information need not be based on the first emotion information and the first biological information. That is, the generation unit 220 may generate the recommended meal information not on the basis of the first emotion information nor the first biological information, but on the basis of the desired emotion information.

The output unit 230 can output the recommended meal information to the user terminal 300.

### 1.3 Configuration of User Terminal 300

The user terminal 300 is realized by using a portable device (e.g., a tablet computer or a smartphone) connectable to the learning system 100 and the information processing system 200 over a communication network (e.g., the Internet). Note that the user terminal 300 may be realized by using a wearable device (e.g., a smartwatch or smart glasses).

The user terminal 300 can receive input of the first emotion information, the first biological information, and the desired emotion information from the user before a meal and can receive input of meal information from the user before the meal, in the middle of the meal, or after the meal. Furthermore, the user terminal 300 can receive input of the second emotion information from the user after the meal. Then, the user terminal 300 can transmit the first emotion information, the first biological information, the meal information, and the second emotion information to the learning system 100 and transmit the first emotion information, the first biological information, and the desired emotion information to the information processing system 200. Furthermore, the user terminal 300 can receive the recommended meal information from the information processing system 200 and display the recommended meal information on a display before a meal.

Note that the user terminal 300 need not receive input of various kinds of information from the user. For example, the user terminal 300 may acquire an image of a dish taken by a camera and acquire meal information by performing object recognition or the like on the image. Furthermore, for example, the user terminal 300 may acquire the first biological information from the biological sensor.

### 1.4 Machine Learning Method

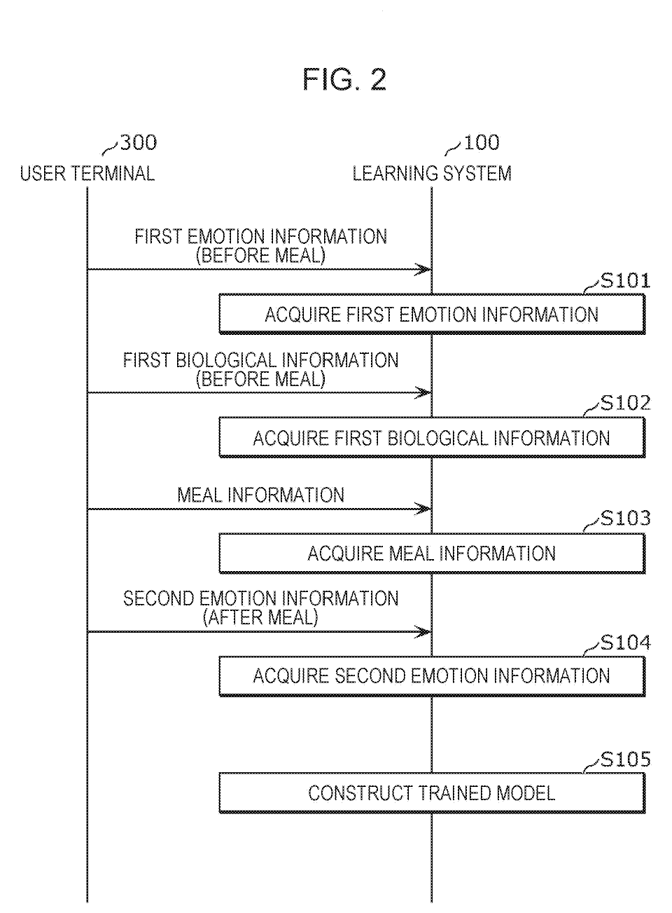

Next, a machine learning method performed by the learning system 100 is described with reference to FIGS. 2 to 6. FIG. 2 is a sequence diagram of a learning phase according to the present embodiment. In the learning phase, machine learning is performed by the learning system 100.

### Step S101

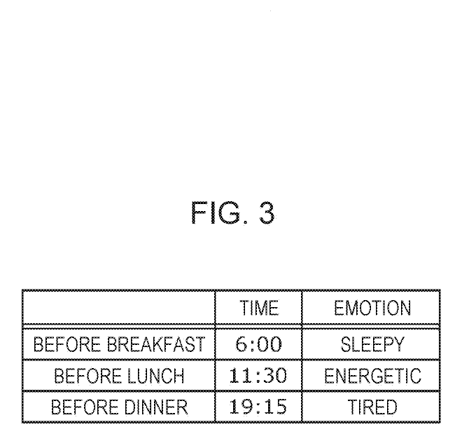

First, the first emotion information acquisition unit 111 acquires the first emotion information concerning a user's emotion before a meal from the user terminal 300. FIG. 3 illustrates an example of the first emotion information according to the present embodiment. In FIG. 3, the first emotion information includes items "time" and "emotion". For example, the first emotion information indicates that a user's emotion at a time "6:00" "before breakfast" is "sleepy". Note that step S101 is optional and may be skipped.

### Step S102

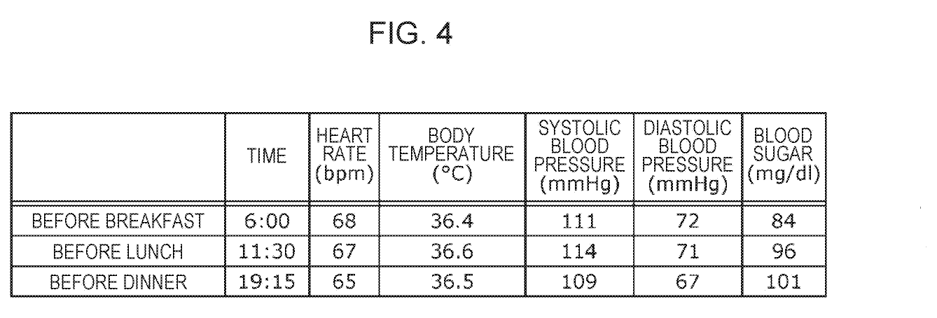

Next, the first biological information acquisition unit 112 acquires the first biological information of the user before the meal from the user terminal 300. FIG. 4 illustrates an example of the first biological information according to the present embodiment. In FIG. 4, the first biological information includes items "time", "heart rate", "body temperature", "systolic blood pressure", "diastolic blood pressure", and "blood sugar". For example, the first biological information indicates that the heart rate is "68 bpm", the body temperature is "36.4 degrees", the systolic blood pressure is "111 mmHg", the diastolic blood pressure is "72 mmHg", and the blood sugar is "84 mg/dl" at a time "6:00" "before breakfast". Note that step S102 is optional and may be skipped.

### Step S103

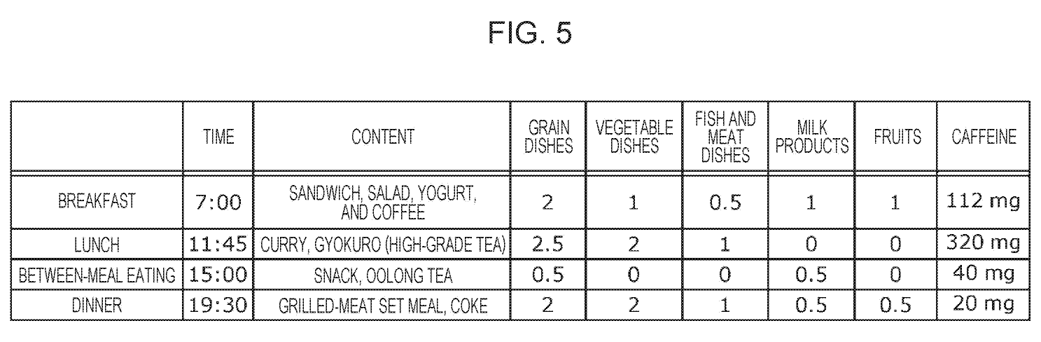

Next, the meal information acquisition unit 113 acquires the meal information concerning content of the meal eaten by the user from the user terminal 300. FIG. 5 illustrates an example of the meal information according to the present embodiment. In FIG. 5, the meal information includes items "time", "content", "grain dishes", "vegetable dishes", "fish and meat dishes", "milk products", "fruits", and "caffeine". For example, the meal information indicates that content of a breakfast eaten at a time "7:00" is "sandwich", "salad", "yogurt", and "coffee", and amounts of "grain dishes", "vegetable dishes", "fish and meat dishes", "milk products", "fruits", and "caffeine" are "2", "1", "0.5", "1", "1", and "112 mg", respectively.

### Step S104

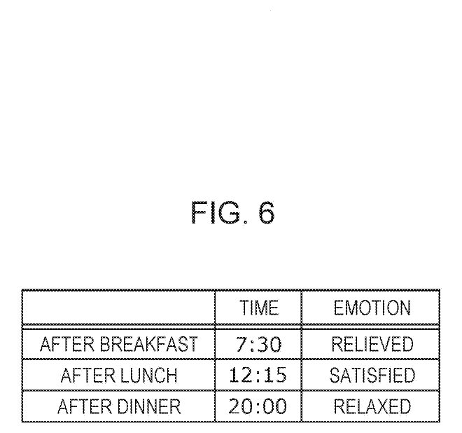

Next, the second emotion information acquisition unit 114 acquires the second emotion information concerning a user's emotion after the meal from the user terminal 300. FIG. 6 illustrates an example of the second emotion information according to the present embodiment. In FIG. 6, the second emotion information includes items "time" and "emotion". For example, the second emotion information indicates that a user's emotion at a time "7:30" "after breakfast" is "relieved".

### Step S105

Finally, the model construction unit 120 constructs the trained model 130 that outputs the recommended meal information on the basis of input of the first emotion information, the first biological information, and the desired emotion information by performing machine learning using a set of the first emotion information, the first biological information, the meal information, and the second emotion information acquired in steps S101 to S104 as a training dataset.

### 1.5 Information Processing Method

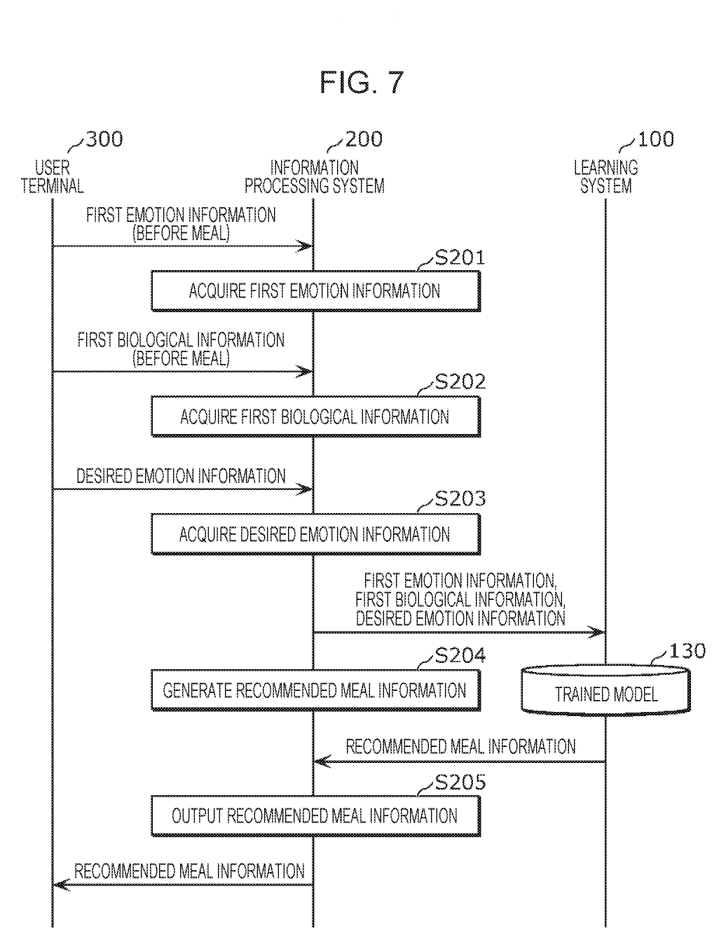

Next, the information processing method performed by the information processing system 200 is described with reference to FIGS. 7 to 10. FIG. 7 is a sequence diagram of a prediction phase according to the present embodiment. In the prediction phase, the information processing system 200 generates (predicts) the recommended meal information by using the trained model 130.

### Step S201

First, the first emotion information acquisition unit 211 acquires the first emotion information concerning a user's emotion before a meal from the user terminal 300. The first emotion information acquired in this step is similar to the first emotion information acquired in step S101 of FIG. 2, and therefore description thereof is omitted. Note that step S201 is optional and may be skipped.

### Step S202

Next, the first biological information acquisition unit 212 acquires the first biological information of the user before the meal from the user terminal 300. The first biological information acquired in this step is similar to the first biological information acquired in step S102 of FIG. 2, and therefore description thereof is omitted. Note that step S202 is optional and may be skipped.

### Step S203

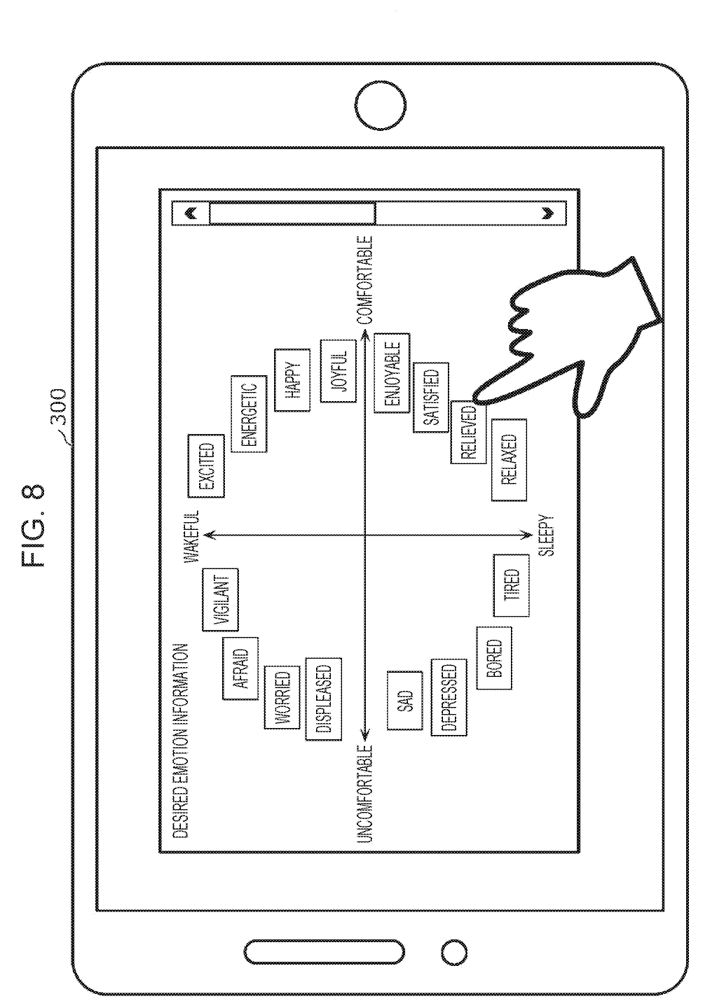

Next, the desired emotion information acquisition unit 213 acquires the desired emotion information concerning an emotion which the use desires to achieve through the meal. FIG. 8 illustrates an example of an input screen for the desired emotion information according to the present embodiment. In FIG. 8, the predetermined mental attributes (excited, energetic, happy, joyful, enjoyable, satisfied, relieved, relaxed, tired, bored, depressed, sad, displeased, worried, afraid, and vigilant) are displayed on a two-dimensional plane constituted by two axes, specifically, an axis indicating comfortable/uncomfortable and an axis indicating wakeful/sleepy on the display of the user terminal 300. The user can input the desired emotion information by touching a mental attribute which the user desires to achieve through the meal from among the mental attributes displayed on the display. Note that a means used to input the desired emotion information is not limited to the graphical user interface (GUI) of FIG. 8. For example, the means used to input the desired emotion information may be a voice user interface (VUI).

### Step S204

Next, the generation unit 220 generates the recommended meal information on the basis of the first emotion information, the first biological information, and the desired emotion information. Specifically, the generation unit 220 generates the recommended meal information by inputting the first emotion information, the first biological information, and the desired emotion information to the trained model 130. That is, the generation unit 220 gives the first emotion information, the first biological information, and the desired emotion information to the trained model 130 and obtains the recommended meal information from the trained model 130.

Step S205

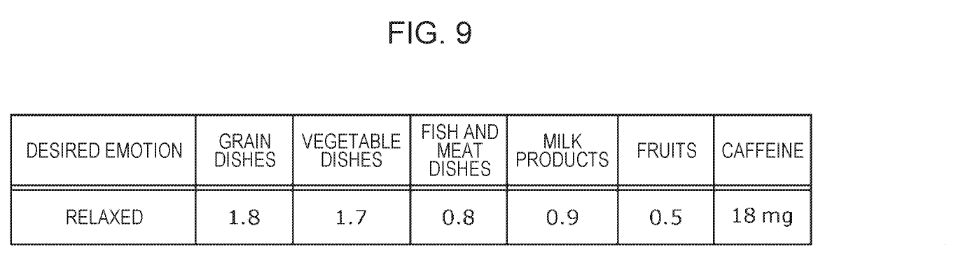

Finally, the output unit 230 outputs the recommended meal information to the user terminal 300. FIG. 9 illustrates an example of the recommended meal information according to the present embodiment. In FIG. 9, the recommended meal information includes items "desired emotion", "grain dishes", "vegetable dishes", "fish and meat dishes", "milk products", "fruits", and "caffeine". For example, the recommended meal information indicates that recommended amounts of the dish categories ("grain dishes", "vegetable dishes", "fish and meat dishes", "milk products", "fruits", and "caffeine") that constitute a meal recommended to achieve a desired emotion "relaxed" are "1.8", "1.7", "0.8", "0.9", "0.5", and "18 mg", respectively.

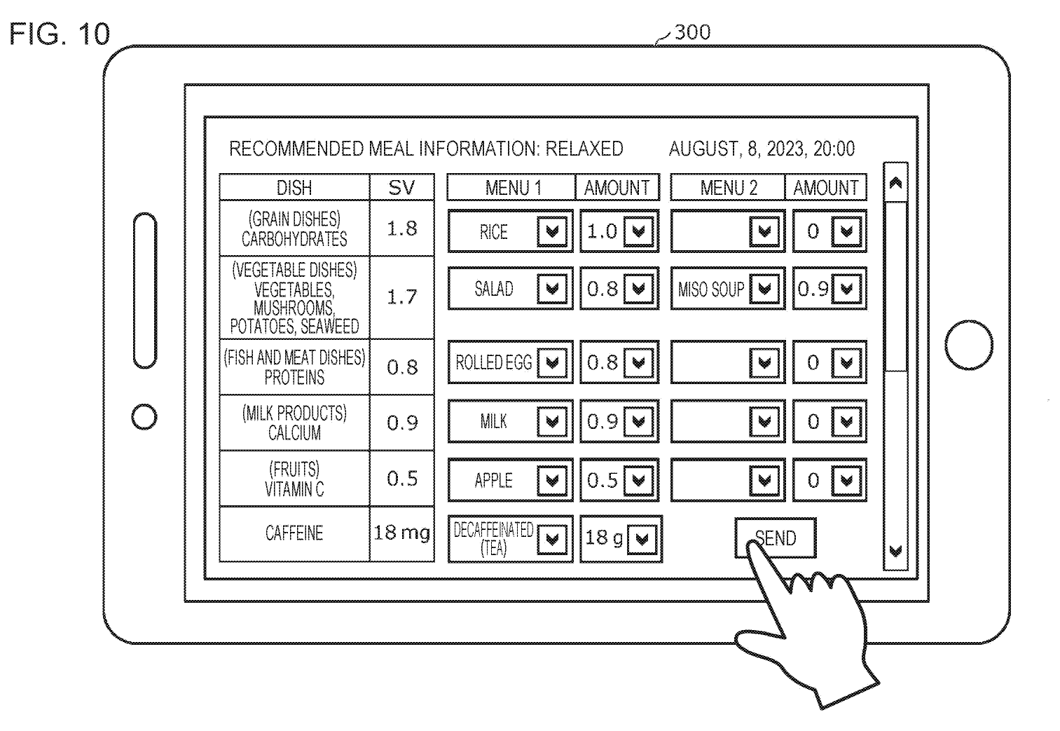

As described above, the recommended meal information output from the information processing system 200 to the user terminal 300 is displayed on the display of the user terminal 300. FIG. 10 illustrates an example of a display screen for displaying the recommended meal information according to the present embodiment. In FIG. 10, recommended amounts (SV) of dish categories that constitute a meal recommended to achieve a desired emotion "relaxed" are displayed on the display of the user terminal 300. Furthermore, one or more dish menu candidates belonging to each of the dish categories are displayed as a dropdown list on the display of the user terminal 300. In FIG. 10, for example, "rice" is selected from among dish menu candidates belonging to the grain dishes, and ""1.0" is selected as an amount thereof. In this way, a dish menu and an amount thereof are set for each of the dish categories and are then transmitted to the learning system 100.

### 1.6 Overview

As described above, according to the information processing method and the information processing system 200 according to the present embodiment, recommended meal information for achieving an emotion desired by a user is generated on the basis of desired emotion information concerning an emotion which the user desires to achieve through a meal. It is therefore possible to provide more suitable recommended meal information to achieve the emotion desired by the user. For example, in a case where meal information is provided on the basis of a user's emotion in the future predicted from schedule information as in Patent Literature 1, there is a possibility that the meal information is provided on the basis of an emotion different from an emotion desired by a user. Therefore, according to the method described in Patent Literature 1, meal information unsuitable for achieving an emotion desired by a user may be undesirably provided. On the other hand, according to the information processing method and the information processing system 200 according to the present embodiment, recommended meal information is generated on the basis of desired emotion information, and therefore more suitable recommended meal information can be provided to achieve an emotion desired by a user.

### Embodiment 2

Next, Embodiment 2 is described. The present embodiment is mainly different from Embodiment 1 in that desired biological information is generated from desired emotion information and recommended meal information is generated from the generated desired biological information instead of generating recommended meal information directly from desired emotion information. The following specifically describes Embodiment 2, mainly differences from Embodiment 1 with reference to the drawings.

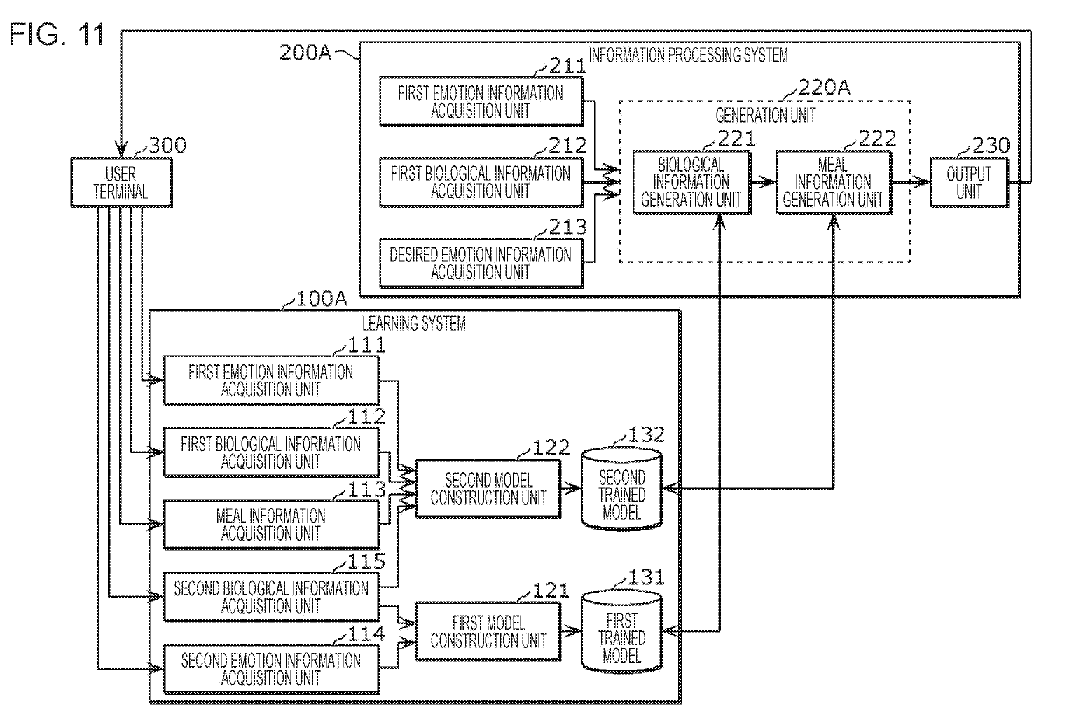

FIG. 11 is a block diagram illustrating a configuration of a learning system 100A and a configuration of an information processing system 200A according to the present embodiment.

### 2.1 Configuration of Learning System 100A

The learning system 100A includes a first emotion information acquisition unit 111, a first biological information acquisition unit 112, a meal information acquisition unit 113, a second emotion information acquisition unit 114, a second biological information acquisition unit 115, a first model construction unit 121, a second model construction unit 122, a first trained model 131, and a second trained model 132.

The second biological information acquisition unit 115 can acquire second biological information of a user after a meal from a user terminal 300. Note that the second biological information acquisition unit 115 may acquire the second biological information from a biological sensor attached to the user. The second biological information is physiological information which the user exhibits after the meal. The second biological information can be, for example, a heart rate, a body temperature, a blood pressure, blood sugar, or any combination thereof, but is not limited to these, as with the first biological information.

The first model construction unit 121 can construct the first trained model 131 by performing machine learning (supervised learning) using a set of second emotion information and second biological information as a training dataset. The first model construction unit 121 can improve prediction accuracy of the first trained model 131 by repeating update of model parameters and evaluation of the model having the updated parameters by using training datasets. Note that an algorithm used for construction of the first trained model 131 is not limited in particular.

The second model construction unit 122 can construct the second trained model 132 by performing machine learning (supervised learning) using a set of first emotion information, first biological information, meal information, and second biological information as a training dataset. The second model construction unit 122 can improve prediction accuracy of the second trained model 132 by repeating update of model parameters and evaluation of the model having the updated parameters by using training datasets. Note that the first emotion information and/or the first biological information are optional and need not be used for construction of the second trained model 132. An algorithm used for construction of the second trained model 132 is not limited in particular.

The first trained model 131 is trained by machine learning using a set of the second emotion information and the second biological information as a training dataset. As a result, the first trained model 131 can output desired biological information corresponding to an emotion which the user desires to achieve through a meal in response to input of desired emotion information concerning the emotion which the user desires to achieve through the meal. That is, in the first trained model 131, the desired emotion information is an explanatory variable, and the desired biological information is an objective variable.

The second trained model 132 is trained by machine learning using a set of the first emotion information, the first biological information, the meal information, and the second biological information as a training dataset. As a result, the second trained model 132 can output recommended meal information in response to input of the first emotion information, the first biological information, and the desired biological information. That is, in the second trained model 132, the desired biological information, the first emotion information (optional), and the first biological information (optional) are explanatory variables, and the recommended meal information is an objective variable.

### 2.2 Configuration of Information Processing System 200A

The information processing system 200A includes a first emotion information acquisition unit 211, a first biological information acquisition unit 212, a desired emotion information acquisition unit 213, a generation unit 220A, and an output unit 230.

The generation unit 220A can generate the recommended meal information on the basis of the first emotion information, the first biological information, and the desired emotion information. Specifically, the generation unit 220A includes a biological information generation unit 221 and a meal information generation unit 222.

The biological information generation unit 221 can generate desired biological information corresponding to an emotion which a user desires to achieve through a meal on the basis of the desired emotion information. Specifically, the biological information generation unit 221 can generate the desired biological information by inputting the desired emotion information to the first trained model 131.

The meal information generation unit 222 can generate the recommended meal information on the basis of the first emotion information, the first biological information, and the desired biological information. Specifically, the meal information generation unit 222 can generate the recommended meal information by inputting the first emotion information, the first biological information, and the desired biological information to the second trained model 132. Note that in the meal information generation unit 222, generation of the recommended meal information need not be based on the first emotion information and the first biological information. That is, the meal information generation unit 222 may generate the recommended meal information not on the basis of the first emotion information nor the first biological information, but on the basis of the desired biological information.

### 2.3 Machine Learning Method

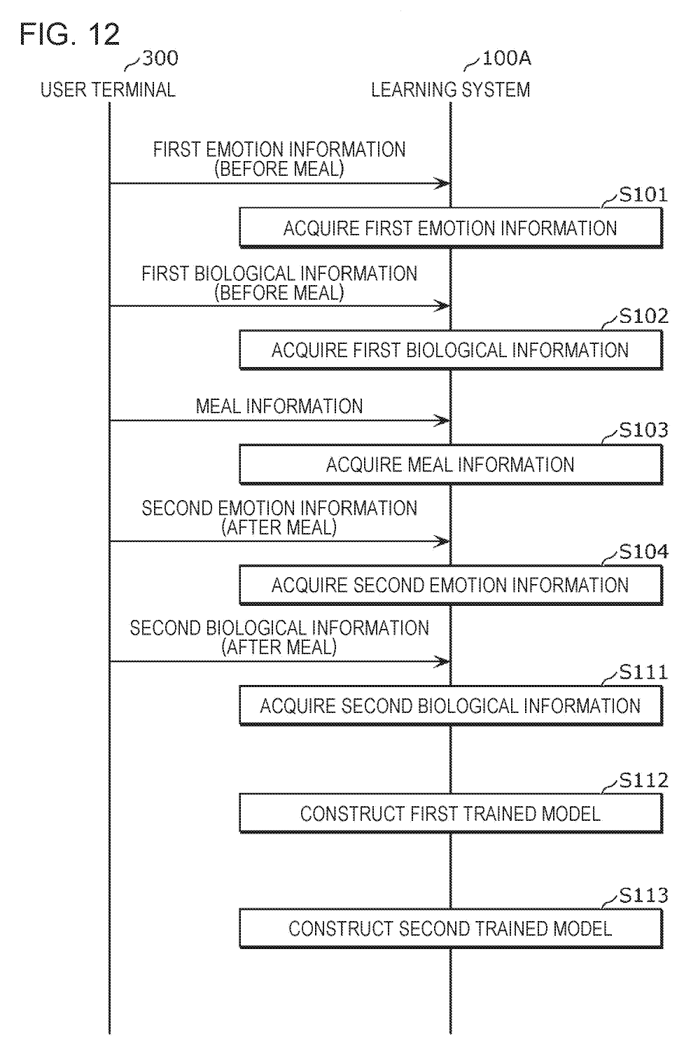

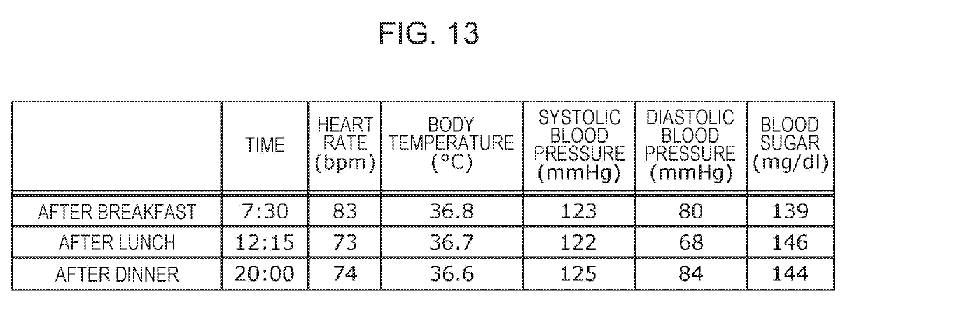

Next, a machine learning method performed by the learning system 100A is described with reference to FIGS. 12 and 13. FIG. 12 is a sequence diagram of a learning phase according to the present embodiment. In the learning phase, machine learning is performed by the learning system 100A.

### Steps S101 to S104

First, the first emotion information, the first biological information, the meal information, and the second emotion information are acquired from the user terminal 300 by the first emotion information acquisition unit 111, the first biological information acquisition unit 112, the meal information acquisition unit 113, and the second emotion information acquisition unit 114, respectively, as in Embodiment 1.

### Step S111

Next, the second biological information acquisition unit 115 acquires the second biological information of a user after a meal from the user terminal 300. FIG. 13 illustrates an example of the second biological information according to the present embodiment. In FIG. 13, the second biological information includes items "time", "heart rate", "body temperature", "systolic blood pressure", "diastolic blood pressure", and "blood sugar". For example, the second biological information indicates that the heart rate is "83 bpm", the body temperature is "36.8 degrees", the systolic blood pressure is "123 mmHg", the diastolic blood pressure is "80 mmHg", and the blood sugar is "139 mg/dl" at a time "7:30" "after breakfast".

### Step S112

Next, the first model construction unit 121 constructs the first trained model 131 that outputs desired biological information on the basis of input of the desired emotion information by performing machine learning using a set of the second emotion information and the second biological information acquired in steps S104 and S111 as a training dataset.

### Step S113

Finally, the second model construction unit 122 constructs the second trained model 132 that outputs recommended meal information on the basis of input of the first emotion information, the first biological information, and the desired biological information by performing machine learning using a set of the first emotion information, the first biological information, the meal information, and the second biological information acquired in steps S101 to S103 and S111 as a training dataset.

### 2.4 Information Processing Method

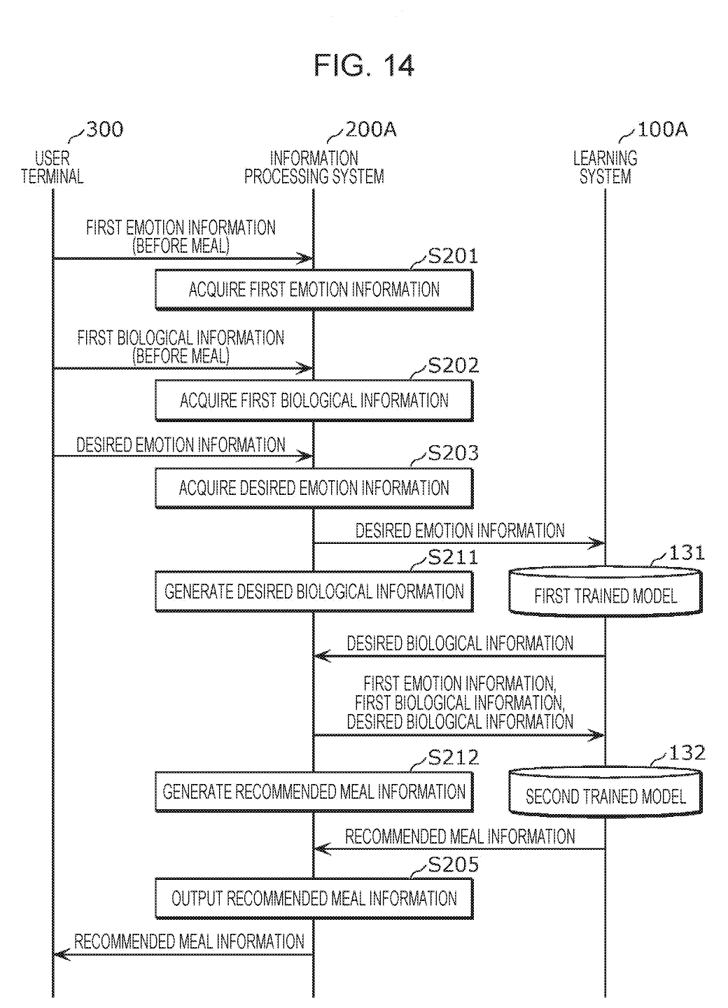

Next, an information processing method performed by the information processing system 200A is described with reference to FIG. 14. FIG. 14 is a sequence diagram of a prediction phase according to the present embodiment. In the prediction phase, the information processing system 200A generates (predicts) recommended meal information by using the first trained model 131 and the second trained model 132.

### Steps S201 to S203

First, the first emotion information, the first biological information, and the desired emotion information are acquired from the user terminal 300 by the first emotion information acquisition unit 211, the first biological information acquisition unit 212, and the desired emotion information acquisition unit 213, respectively, as in Embodiment 1.

### Step S211

Next, the biological information generation unit 221 generates the desired biological information on the basis of the desired emotion information. Specifically, the biological information generation unit 221 generates the desired biological information by inputting the desired emotion information to the first trained model 131. That is, the biological information generation unit 221 gives the desired emotion information to the first trained model 131 and obtains the desired biological information from the first trained model 131.

### Step S212

Next, the meal information generation unit 222 generates the recommended meal information on the basis of the first emotion information, the first biological information, and the desired biological information. Specifically, the meal information generation unit 222 generates the recommended meal information by inputting the first emotion information, the first biological information, and the desired biological information to the second trained model 132. That is, the meal information generation unit 222 gives the first emotion information, the first biological information, and the desired biological information to the second trained model 132 and obtains the recommended meal information from the second trained model 132.

### Step S205

Finally, the recommended meal information is output to the user terminal 300 by the output unit 230, as in Embodiment 1.

### 2.5 Overview

As described above, according to the information processing method and the information processing system 200A according to the present embodiment, desired biological information is generated on the basis of desired emotion information, and recommended meal information is generated on the basis of the desired biological information. By thus generating desired biological information from desired emotion information and then generating recommended meal information from the desired biological information, not only an emotion consciously desired by a user, but also an emotion subconsciously desired by the user can be reflected in the recommended meal information, and recommended meal information more suitable for an emotion desired by the user can be provided.

### Other Embodiments and Modifications

Although the information processing system, the information processing method, the learning system, and the learning method have been described above on the basis of the embodiments, the information processing system, the information processing method, the learning system, and the learning method according to the present disclosure are not limited to the above embodiments. Other embodiments realized by combining any of the constituent elements described in the above embodiments, as well as various modifications that may be conceived by those skilled in the art without departing from the spirit of the present disclosure, are also encompassed within the scope of the present disclosure.

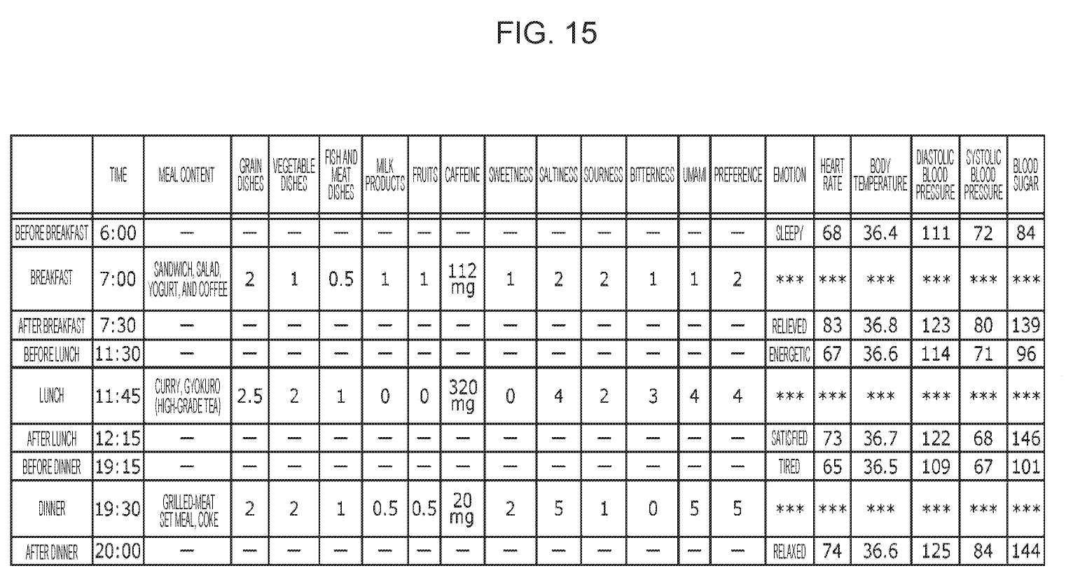

For example, a training dataset used in machine learning is not limited to the datasets described in the above embodiments. For example, the training dataset may include sensory evaluation values of a meal or may include emotion information and/or biological information during a meal. Such a modification of the training dataset is specifically described with reference to FIG. 15. FIG. 15 illustrates an example of a training dataset according to a modification. In FIG. 15, the training dataset includes a set of meal information, first emotion information, first biological information, second emotion information, and second biological information. The meal information includes items "sweetness", "saltiness", "sourness", "bitterness", "umami", and "preference" in addition to the items of FIG. 5. Sensory evaluation values are input to the items "sweetness", "saltiness", "sourness", "bitterness", "umami", and "preference". Although "***" is shown as emotion information and biological information during a meal in FIG. 15, a value may be input.

An expression "at least one of a first element or a second element" may be interpreted as "the first element, the second element, or a combination of the first element and the second element".

The present disclosure is applicable to an information processing system that outputs recommended meal information concerning content of a meal recommended for a user to achieve an emotion which the user desires to achieve through a meal.

What is claimed is:

1. An information processing method performed by a computer, the information processing method comprising:
acquiring desired emotion information concerning a psychological state which a user aims to achieve after a meal;
generating recommended meal information concerning content of a meal recommended for the user to achieve the psychological state on a basis of the desired emotion information; and
outputting the recommended meal information thus generated.

2. The information processing method according to claim 1, wherein
in the generating the recommended meal information, the recommended meal information is generated by inputting the desired emotion information to a trained model, and
the trained model has been trained by machine learning using a set of meal information concerning content of a meal eaten by the user and second emotion information concerning an emotion of the user after the meal as a training dataset.

3. The information processing method according to claim 1, further comprising acquiring at least one of first emotion information concerning an emotion of the user before the meal or first biological information of the user before the meal, wherein
in the generating the recommended meal information, the recommended meal information is generated on a basis of at least one of the first emotion information or the first biological information in addition to the desired emotion information.

4. The information processing method according to claim 3, wherein
in the generating the recommended meal information, the recommended meal information is generated by inputting the desired emotion information and at least one of the first emotion information or the first biological information to a trained model, and
the trained model has been trained by machine learning using a set of meal information concerning content of a meal eaten by the user, second emotion information concerning an emotion of the user after the meal, and at least one of first emotion information concerning an emotion of the user before the meal or first biological information of the user before the meal as a training dataset.

5. The information processing method according to claim 1, wherein
the generating the recommended meal information includes generating desired biological information corresponding to the psychological state on a basis of the desired emotion information and generating the recommended meal information on a basis of the desired biological information.

6. The information processing method according to claim 5, wherein
in the generating the desired biological information, the desired biological information is generated by inputting the desired emotion information to a first trained model, and
the first trained model has been trained by machine learning using a set of emotion information concerning an emotion of the user and biological information of the user corresponding to the emotion information as a training dataset.

7. The information processing method according to claim 5, wherein
in the generating the recommended meal information, the recommended meal information is generated by inputting the desired biological information to a second trained model, and
the second trained model has been trained by machine learning using a set of meal information concerning content of a meal eaten by the user and second biological information of the user after the meal as a training dataset.

8. The information processing method according to claim 5, further comprising acquiring at least one of first emotion information concerning an emotion of the user before the meal or first biological information of the user before the meal, wherein
in the generating the recommended meal information, the recommended meal information is generated on a basis of at least one of the first emotion information or the first biological information in addition to the desired biological information.

9. The information processing method according to claim 8, wherein
in the generating the recommended meal information, the recommended meal information is generated by inputting the desired biological information and at least one of the first emotion information or the first biological information to a second trained model, and
the second trained model has been trained by machine learning using a set of meal information concerning content of a meal eaten by the user, second biological information of the user after the meal, and at least one of first emotion information concerning an emotion of the user before the meal or first biological information of the user before the meal as a training dataset.

10. The information processing method according to claim 1, wherein
in the outputting the recommended meal information, a recommended amount of each of dish categories that constitute the meal is displayed as the recommended meal information on a display.

11. The information processing method according to claim 10, wherein
in the outputting the recommended meal information, one or more dish menu candidates belonging to each of the dish categories are displayed, and
the information processing method further comprises receiving, from the user, selection of the displayed one or more dish menu candidates and input of an amount of each of the selected one or more dish menus.

12. A non-volatile recording medium storing a program for causing a computer to perform the information processing method according to claim 1.

13. An information processing system comprising:
an acquirer that acquires desired emotion information concerning a psychological state which a user aims to achieve after a meal;
a generator that generates recommended meal information concerning content of a meal recommended for the user to achieve the psychological state on a basis of the desired emotion information; and
an outputter that outputs the recommended meal information thus generated.

14. A learning system comprising:
a meal information acquirer that acquires meal information concerning content of a meal eaten by a user;
a biological information acquirer that acquires biological information of the user after the meal;
an emotion information acquirer that acquires emotion information concerning an emotion of the user after the meal;
a first model constructor that constructs a first trained model that outputs desired biological information corresponding to a psychological state which the user aims to achieve after a meal on a basis of input of desired emotion information concerning the psychological state by performing machine learning using a set of the emotion information and the biological information as a training dataset; and
a second model constructor that constructs a second trained model that outputs recommended meal information recommended for the user to achieve the desired biological information on a basis of input of the desired biological information by performing machine learning using a set of the biological information and the meal information as a training dataset.

15. The information processing method according to claim 1, wherein
the generating the recommended meal information is performed by using a model that represents a state transition from a state before a meal to a state after the meal on a basis of biological information and emotion information of the user before a meal and the desired emotion information.
